(19) United States
(12) Patent Application Publication
(10) Pub. No.: US 2009/0320559 A1
(43) Pub. Date: Dec. 31, 2009

(54) Evaluation of the Fragrance Characteristics of a Substance

(75) Inventors: Marie Josephine Louise Lemieuvre, Paris (FR); Michel Girard, Paris (FR); Jerome Jallat, Suresnes (FR); Tahir Iftikhar Malik, Stokesley (GB)

Correspondence Address: JONES DAY, 222 EAST 41ST ST, NEW YORK, NY, 10017, US

(73) Assignee: Imperial Chemical Industries PLC, London (GB)

(21) Appl. No.: 12/083,772

(22) PCT Filed: Oct. 17, 2006
(86) PCT No.: PCT/GB2006/003865

§ 371 Date: Mar. 9, 2009

(30) Foreign Application Priority Data
Oct. 18, 2005 (GB) 0521119.8

(51) Int. Cl. G01N 33/00

(52) U.S. Cl. 73/23.34; 73/863.11

(57) ABSTRACT

Apparatus for evaluating the fragrance characteristics of a substance comprises a cylindrical chamber (2) in which is located a block (3) for supporting a substance, e.g. a fragrance to be evaluated. A flow of air, generated by a fan (4), passes through the chamber (2), around the substance and leaves the chamber via a main outlet (7) and a plurality of sensing outlets (12) each provided with a metering valve (13) leading the air flow to a corresponding nasal mask (16). Individuals take two or three sniffs of the air flow from the mask and record their findings on a pre-prepared form.

### FIELD OF THE INVENTION

This invention relates to an apparatus for, and a method of, evaluating the fragrance characteristics of a substance, for example in the development of a fine fragrance (made up of a mixture of many perfume ingredients) or a fragrance for a consumer product such as a cosmetic, hairspray or air freshener, a cleaning product or a food product. The invention also relates to a method of formulating a fragrance, and the resulting fragrance.

### BACKGROUND OF THE INVENTION

There are three main criteria in the development of a fine fragrance. These can be classified as emotional parameters and rational parameters. The first emotional parameter is hedonism or liking and consumer research can be used to answer this question. The second emotional parameter is memorability or signature of the fine fragrance, i.e., how easy can it be recognised and there are methodologies available to be able to work this out. The missing link is the easy determination of the physical performance of the fine fragrance, that is the perceived intensity. This can be defined with reference to the position of the source relative to the sensor and whether the source is stationary or moving and the distance between the two. The environment in terms of whether the scenario being considered is an indoor scenario or an outdoor scenario will have an impact on the trail presented. The essential steps in the presentation of the fragrance are emission from the source by evaporation into the air, the transmission to sensor location through the air by diffusive and convective means and by the sensor inhaling and registering the sample in the olfactory system. The invention aims to reproduce these steps.

### SUMMARY OF THE INVENTION

According to one aspect the invention provides apparatus for evaluating the fragrance characteristics of a substance, the apparatus comprising a chamber, a support in the chamber for supporting a sample of the substance, a device for generating a flow of air in the chamber, a plurality of sensing outlets from the chamber, and means for controlling air flow rate, in use the device generating a flow of air through the chamber and through at least one sensing outlet, the flow from which is used to evaluate the fragrance characteristics of the sample.

Preferably, the controlling means comprises a power supply to the device. Further preferably, the controlling means also or instead comprises a flow control valve in each sensing outlet. The apparatus may further comprise a main outlet for egress of air from the chamber, in which case the controlling means preferably also comprises a valve in the main outlet. The device is preferably a fan. The plurality of sensing outlets, may be positioned symmetrically with respect to the position of the sample on the support, so that the fragrance characteristics can be made substantially uniform in each of the plurality of sensing outlets. In a preferred embodiment, each sensing outlet comprises a flexible tube terminating in a mask for inhalation by an individual.

The support for the substance sample may be equipped with a heater (which may be controllable) for heating the sample. This enables the conditions of skin temperature to be simulated, and also enables the process of fragrance transfer to the air flow to be hastened.

According to another aspect of the invention there is provided a method of evaluating the fragrance characteristics of a substance, comprising placing a sample of the substance in a chamber, passing a flow of air in the chamber around the sample and out of the chamber through at least one of a plurality of sensing outlets, controlling air flow rate, and using air flow through at least one sensing outlet to evaluate the fragrance characteristics of the sample.

The substance under test may be a fine fragrance (comprising a mixture of many perfume materials with optional diluent), or other material with fragrance properties such as a fragrance or perfume for a consumer product such as a cosmetic product, a cleaning product etc, or it may be a food product, the olfactory characteristics of which are of interest.

The apparatus can be set up to simulate a chosen one of a number of possible practical situations, including indoors, outdoors (with or without wind), fragrance source stationary or moving, human sensor stationary or moving, remanance of fragrance source or air temperature. To simulate a particular situation, the following key parameters can be adjusted: sample temperature, quantity and surface area of sample, vapour pressure, air flow rate and air flow temperature, and mixing imparted within the chamber due to flow pattern and convective fluxes from hot surfaces, e.g the support.

The invention also includes within its scope a method of formulating a desired fragrance by repeatedly carrying out the method of the invention with substances of differing compositions and selecting the desired fragrance on the basis of the evaluation of the fragrance characteristics of the substances. Each substance may be a potential desired fragrance and the selection is carried out by a method which includes electing one of the potential fragrances to be the desired fragrance. Alternatively, or in addition, each substance may be a potential ingredient or component of the desired fragrance and the selection is carried out by a method which includes combining selected potential ingredients or components to form the desired fragrance. The invention also covers the resulting fragrance formulated by the method.

In the preferred embodiment, the pressure drop to flow characteristics through the system is such that changes in the flow rates through any of the outlets (main or sensory) e.g. as obtained when manipulating the valves, have very little influence on the stability or flow rate through any of the other lines. The amount of convection air available for mixing within the chamber may be varied by placing additional impediment objects in the flow path or hot surfaces that help generate convection.

The air flow to the main outlet may be varied in order to simulate the air change rate in a real-life indoor scenario.

Mathematical models comprising thermodynamics and multi-component mass transfer terms in both the condensed and vapour phases may be used to correlate the performance of the apparatus to that of a real-life scenario. The mathematical models can predict the influence of temperature on the performance of the apparatus, and thus can be used through temperature dependent correlation, to modify the rate at which the test experiment proceeds.

Simultaneous analytical and sensory measurement may be carried out, with some of the sensory lines being used by human panellists while the other lines are used for trapping for analysis.

Sensory measurements and the physical measurements are correlated with each other and with the dynamic mathematical models to gain insights into the trailing behaviour of fragrances and through choice of experiments on model formulations to the interactions between fragrance components with regard to intensity and trail-generating properties.

The fragrance sample may be placed within the chamber by the operator only for a brief period (e.g 2 minutes) and then removed, with or without the knowledge of the panellist, for the determination of the ‘transient’ intensity profile.

Chambers of different sizes (from about 30 litres to 3 m³) may be used to provide additional data for correlating the impact of scale and mixing on the fragrance intensity and fragrance-trailing behaviour, for example, perfumers and their evalvators, may subject the same fragrance to tests at three different scales.

### BRIEF DESCRIPTION OF THE DRAWINGS

Two preferred embodiments of the invention will now be described, by way of example, with reference to the accompanying drawings in which:

FIG. 1 is a schematic illustration of apparatus of the first embodiment,

FIG. 2 is an isometric representation of the apparatus of the second embodiment, and

FIG. 3 shows a form used to record results and observations in a test procedure.

### DETAILED DESCRIPTION OF THE DRAWINGS

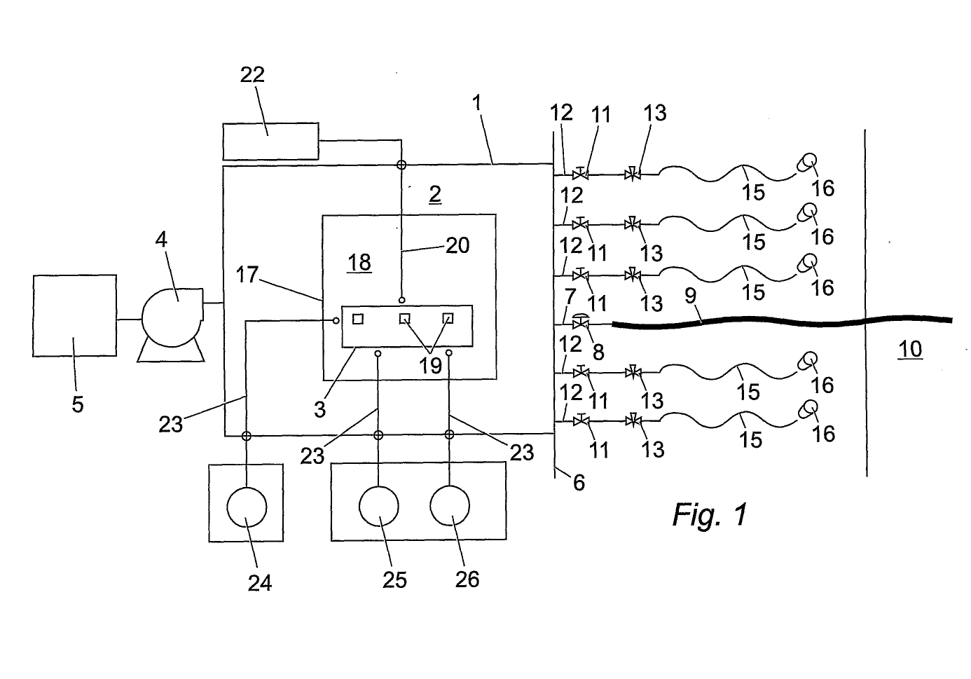

The apparatus of FIG. 1 comprises a stainless steel (or other inert material) cylinder 1 which is positioned with its central axis horizontal and which defines a cylindrical chamber 2 accommodating an aluminium block 3 used to support a sample of a substance (e.g. a perfume) the fragrance characteristics of which are to be evaluated. One circular end (the left hand end as viewed in FIG. 1) has a central inlet aperture through which an inlet flow of air is delivered by a fan 4 (or pump or compressor) powered by an electrical power supply 5, normally an AC mains supply. At its other end, the cylinder 1 is closed by a circular end flange 6 with a central outlet aperture connected to an outlet pipe or line 7 leading to a gate valve 8 for controlling the flow rate of air leaving the cylinder 1 through the central outlet. A length of PTFE-lined stainless steel hose 9 leads the air from the outlet of the gate valve 8 to a fume cupboard 10.

The end flange 6 has five further outlets 12 forming sensing outlets or lines respectively connected by tubing to five on/off ball valves 11 which are connected to five controllable flow control or metering needle valves 13. A length of PTFE-lined stainless steel flexible hose 15, preferably 6 mm diameter, is attached to the outlet of each metering valve 13, so that there are in all five lengths of hose 15 each of which terminates in a corresponding Teflon-coated nasal mask 16. The five sensing outlets 12 in the flange 6 are angularly spaced around the main outlet line 7 so that the five sensing outlets 12 are symmetrical with respect to, and equidistant from, the position of the sample on the support block 3. As a consequence, the fragrance intensity in the air flows through the five sensing outlets 12 is substantially the same at any time. Individual sensing lines can be opened or closed by use of the on/off valves 11 and the flow rate through each individual line can be adjusted by the metering valves 13, and adjustment of the flow rate through any one line has a minimum effect on the flow through other sensing lines. Also, the main exit valve 8 can be controlled to vary the air flow rate from zero to a large volumetric flow rate, again with little impact on the flow through any open sensing line.

Part of the cylindrical wall of the cylinder 1 has a generally rectangular aperture closable by a stainless steel access door 17 which incorporates a transparent viewing window 18 and the periphery of which can be sealed against the edges of the aperture to prevent air leakage at the door position. For the same purpose, the flange 6 is sealed against the end of the cylinder 1.

The chamber 2 is equipped with various control and monitoring devices, including cartridge heaters 19 for heating the block 3 on which the sample is placed, an anemometer probe 20 linked to an air velocity and temperature reader 22 and three thermocouples 23 respectively connected to a temperature reader 24, a controller 25 and a trip 26. The heaters 19 can be used to maintain the sample of a temperature close to human skin temperature, in order to simulate fragrance or scent on the human skin, or the sample can be heated above this temperature to increase the speed of the test procedure.

The detailed operating procedure will now be described. It involves three main steps:

1. The calibration of the flows: to set the flow conditions over the perfume sample to simulate real-life conditions and to set the flow suitable to carry out sensory analysis. This is carried out by the person designated as the operator.

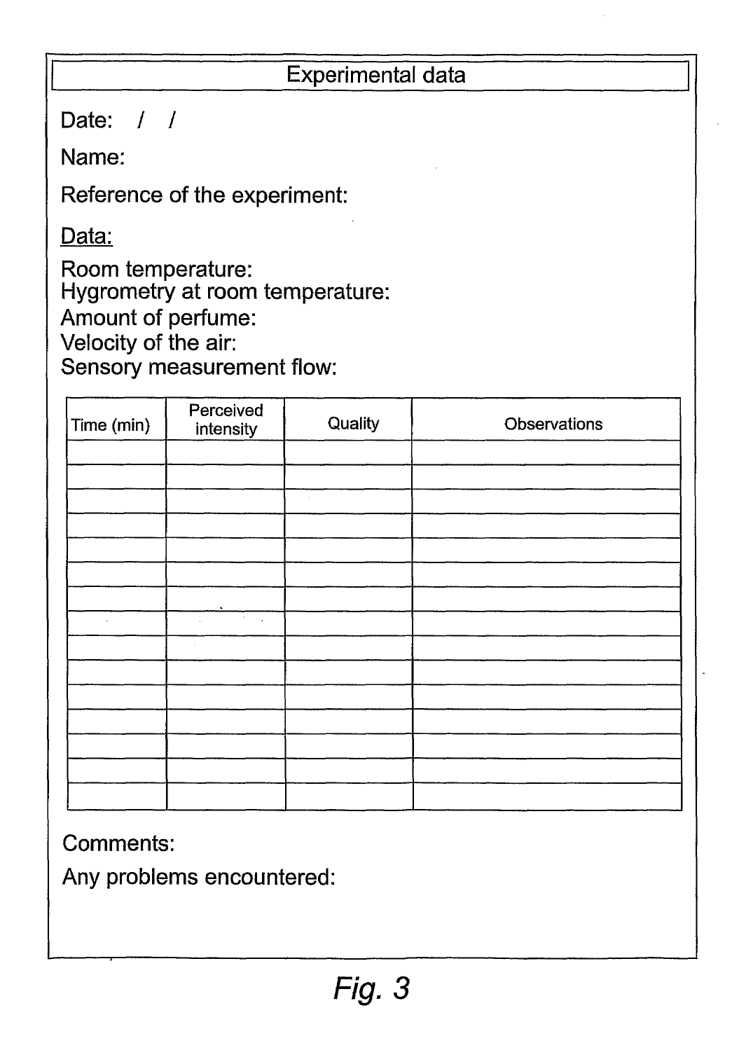

2. The test or evaluation itself: where a group of individuals (the panellists) carry out sensory measurements against time and generate a record on the form of FIG. 3, this record being called a trail profile (intensity and/or quality versus time).

3. The cleaning or decontamination of the apparatus after use to ensure that the next test or experiment will be carried out without any residual perfume in the chamber. Normally, purging the apparatus with cold air is sufficient (but hot air can be used) and dismantling and chemical cleaning can be employed if necessary. This is carried out by the person designated as the operator. The components of the apparatus which are subjected to perfumed air are made of materials (e.g. stainless steel, PTFE, glass) which are inert and thus will not absorb fragrance.

### Setting of the Flows

There are two different flow rates that can be set independently from each other. The first one is the total throughput of air in the chamber 2 and the second one is the flow rate obtained on a given sensing exit line. The total flow rate depends upon a combination of the power input to the fan 4 and the settings of all the exit valves 8 and 13. The fan 4 is selected to enable reasonable flow rates to the sensing lines when the valves 13 on these lines are substantially open.

A recommended procedure for a given fan that has been previously tested is to keep the electrical power applied to a medium and same level from one experiment to the next.

(a) Decide on the value of the flow to be applied through the chamber 2. For example: 50 L/min. This will still lead to a relatively high air change rate if a small size chamber is used.

(b) The main gate valve 8 is fully open. The ball valves 11 are closed. The needle valves 13 are open. Switch on the fan 4 using the power supply.

(c) Tune the power provided to the fan 4 to obtain the right flow into the chamber 2.

(d) Place a flowmeter at the exit of the sampling line to be used to carry out sensory measurements. Switch it on.

(e) Open the ball valve 11 fully. Tune/close gradually the needle valve 13 opening to obtain a flow of about 1 litre/min at the exhaust of the sensing line. This value of 1 l/min has been advised as a suitable flow value to carry out sensory measurement without having the influence of the flow on the measurements. Close the ball valve 11. This way, next time that the ball valve 11 is opened, flow coming out will be suitable for sensory analysis and will be in a repeatable condition.

(f) Similarly the velocity profile in the exhaust line can be measured with these fan and valve settings (both with all sensory lines closed or with some of them open). The flow estimated from the exit lines plus the total flow in the sensing lines in the air travelling through the chamber 2.

Once reasonable settings have been obtained then calibration may not be needed from one experiment to the next. However, the actual air change rate can still be changed by operating the exhaust gate valve 8 and the flow estimated from an anenometer measurement if required.

### The Evaluation

After setting of the apparatus, the test or evaluation involves placing the sample (e.g. of perfume) on a watch glass which is then placed on the support in the chamber 2, using the access door 17 which is subsequently closed and sealed. The fan 4 is energised to generate a flow of air into the chamber 2, around the sample and out of the main outlet and one or more sensing outlets. The air flow through the chamber 2 can be controlled by controlling the power applied to the fan 4 in combination with control of the main valve 8 and one or more of the sensing line valves 11 and 13. The fragrance characteristics of the sample are evaluated in a human testing procedure. A plurality of individuals (up to five in number) are used in the test. Each individual opens the corresponding ball valve 11, places his or her nose in the mask 16 and sniffs two or three times, closes the valve and records the test results and observations on the form shown in FIG. 3. In this manner, only a very small amount of perfumed air needs to enter the room when the test is carried out, it being understood that the flow through the main valve exhausts to the fume cupboard 10. The individuals can carry out the evaluation simultaneously or sequentially.

Individuals differ in the response to a fragrance, but employing a panel of individuals testing identical physical fragrance intensities gives a more representative, and therefore more useful, overall perceived intensity and quality test result. The individuals may all carry out the test simultaneously or at different times, the latter being possible because altering or terminating the flow through one sensing line hardly alters the flow through any other sensing lines that may be open.

Instead of, or in addition to, employing the subjective response of individuals the apparatus can be used in an analytical procedure where air from a sensing line is passed over an absorbent material for a specified time duration after which the absorbent material is analysed.

In order to formulate a desired fragrance, substances which are potential ingredients or components of the desired fragrance can be evaluated by repeatedly carrying out the method previously described, and components selected and then combined in particular proportions to derive the desired fragrance, optionally with one or more evaluations of the combined components. Alternatively, substances which are themselves potential fragrances can be evaluated as previously described and one or more fragrances selected on the basis of the evaluations.

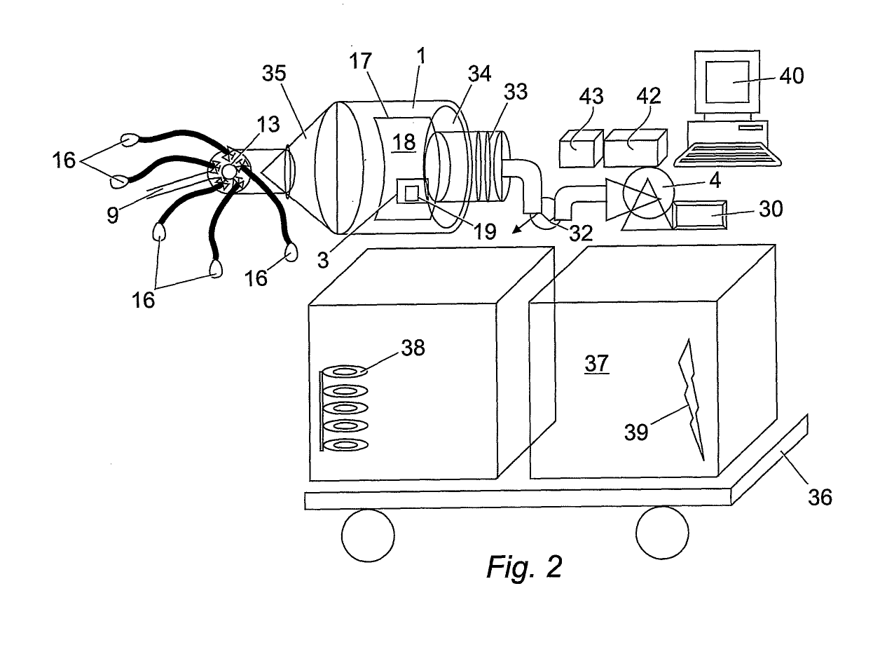

FIG. 2 shows a more developed form of the apparatus, and parts corresponding to those of FIG. 1 bear the same reference numerals.

In FIG. 2, the general direction of airflow is from right to left. A stainless steel cylinder 1 has an access door 17 with a transparent window 18 to view the chamber which again has a support on which is placed a watch glass holding the sample to be evaluated. A fan 4 (or compressor) delivers a flow of air to the chamber 2, the flow being variable in a step-less manner under the control of a controller 30. Air from the fan 4 flows over a heat exchanger 32 to control the temperature of the airflow which passes through filters 33 before entering the chamber through a diffuser 34 to create an evenly distributed flow of air. After passing around the sample, the airflow enters a frusto-conical outlet end 35 of the chamber where the air velocity is increased and its pressure decreased. After this, the air enters a short tube terminating in a central main outlet, leading through a main outlet valve and main outlet line, and also through five sensing outlets 12 leading through five respective metering valves 13 and five sensing lines having respective nasal masks 16. The components of the apparatus thus far described are carried on a trolley 36 for compactness and ease of transport. The space in the base of the trolley is used to accommodate water tanks 37 for holding water for the heat exchanger 32, a chiller 38 for cooling the water and a heater 39 for heating the water. The functioning components are under the control of a computer 40 linked to a programmable logic control unit 42 which in turn is linked to a conditioning box 43 to control the sample heater.

In the apparatus of FIG. 1 or 2, typically quantitative values are as follows:

1. The chamber volume is between 30 litres and about 3 cubic metres representing a scale-down ratio from a ‘typical’ room of 1000 times to 10 times.

2. The chamber air change rate ranges from 0 to ‘normal’ value to ‘maximum’ value. The normal value is based on 2 chamber volume changes per hour. The maximum value is 100 times that. The normal value simulates a typical well ventilated indoor room, while the maximum value simulates outdoor scenarios and is also used for decontamination duties. Thus for a 30 litre chamber volume, the normal value is 1 litre per minute and the maximum value is 100 litre per minute, and for a 3 cubic metre chamber volume, the normal value is 100 litre per minute and the maximum value is 10 cubic metre per minute.

3. Sensory line flow rate ranges from 0 to 1 litre per minute for each line irrespective of chamber volume, with the normal value being 50 ml per minute.

4. The amount of perfume varies from one drop to about 5 g of material with a typical value of 0.05 g used for the 30 litre size chambers. Larger amounts will be used for the larger chamber volumes.

5. The perfume surface areas depend upon the amount of perfume as well as the shape of the container (e.g. more surface is created when a petri dish is placed upside down (concave rather than convex surface up)). The ranges are less than 1 cm² to 100 cm². Larger surface areas will typically be used with larger chamber volumes.

6. The humidity values will range from dry, i.e. 0%, to saturated, i.e. 100%, relative humidity.

7. The sample temperature will range from room temperature to about 45° C., with the normal temperature being skin surface temperature (28° C. to 32° C.).

8. The air temperature will range from room temperature to about 70° C. for decontamination duties.

9. The number of panellists will range from 0 to 5 in the current design. Additional panellists can also be accommodated by redesigning the flange with the exit ports.

10. The number of analytical traps will range from 0 to 5 in the current design. Additional numbers can be accommodated by redesigning the flange with the exit ports.

1. Apparatus for evaluating the fragrance characteristics of a substance, the apparatus comprising a chamber, a support in the chamber for supporting a sample of the substance, a device for generating a flow of air in the chamber, a plurality of sensing outlets from the chamber, and means for controlling air flow rate, in use the device generating a flow of air through the chamber and through at least one sensing outlet, the flow from which is used to evaluate the fragrance characteristics of the sample.

2. Apparatus according to claim 1, wherein the controlling means comprises a power supply to the fan.

3. Apparatus according to claim 1, wherein the controlling means comprises a flow control valve in each sensing outlet.

4. Apparatus according to claim 1, further comprising a main outlet for egress of air from the chamber.

5. Apparatus according to claim 4, wherein the controlling means comprises a valve in the main outlet.

6. Apparatus according to claim 1, wherein the device is a fan.

7. Apparatus according to claim 1, wherein the plurality of sensing outlets are positioned symmetrically with respect to the position of the sample on the support.

8. Apparatus according to claim 1, wherein each sensing outlet comprises a flexible tube terminating in a mask for inhalation by an individual.

9. Apparatus according to claim 1, wherein the support is equipped with a heater for heating the sample.

10. Apparatus according to claim 1, wherein the chamber is defined by a cylinder positioned with its central axis horizontal, the device generating a flow of air into one end of the cylinder, the other end of which is provided with the sensing outlets.

11. Apparatus according to claim 10, wherein the cylinder has a removable access door, sealable against the wall of the cylinder, for access to the support for placing of the sample in the chamber and subsequent removal therefrom.

12. Apparatus according to claim 10, wherein the or each sensing outlet has an on/off valve.

13. A method of evaluating the fragrance characteristics of a substance, comprising placing a sample of the substance in a chamber, passing a flow of air in the chamber, around the sample and out of the chamber through at least one of a plurality of sensing outlets, controlling air flow rate, and using air flow through at least one sensing outlet to evaluate the fragrance characteristics of the sample.

14. A method according to claim 13, wherein the air flow from the sensing outlet is inhaled by an individual who subjectively evaluates the fragrance characteristics.

15. A method according to claim 14, wherein the individual's evaluation of the fragrance characteristics is recorded on a prepared form.

16. A method according to claim 14, wherein a plurality of individuals respectively inhale the air from the plurality of sensing outlets, either simultaneously or at different times.

17. A method formulating a desired fragrance by repeatedly carrying out the method of claim 14 with substances of differing compositions and selecting the desired fragrance on the basis of the evaluation of the fragrance characteristics of the substances.

18. A method according to claim 17, wherein each substance is a potential desired fragrance and the selection is carried out by a method which includes electing one of the potential fragrances to be the desired fragrance.

19. A method according to claim 17, wherein each substance is a potential ingredient or component of the desired fragrance and the selection is carried out by a method which includes combining selected potential ingredients or components to form the desired fragrance.

20. A fragrance formulated by a method according to claim 17.

21. A method of reporting apparatus for evaluating the fragrance characteristics of a substance, the apparatus comprising a chamber, a support in the chamber for supporting a sample of the substance, a device for generating a flow of air in the chamber and a plurality of outlets through which air from the chamber may flow, the method comprising controlling the air flow rate such that a change in the flow rate through any of the outlets has no influence on the flow rate through any of the other outlets.
